(19) United States
(12) Patent Application Publication
(10) Pub. No.: US 2017/0071724 A1
(43) Pub. Date: Mar. 16, 2017

(54) TRANSOSSEOUS SUTURE CONSTRUCTS

(71) Applicant: KATOR, LLC, Logan, UT (US)

(72) Inventors: T. Wade Fallin, Hyde Park, UT (US); Joel Helgerson, Erie, CO (US)

(73) Assignee: KATOR, LLC, Logan, UT (US)

(21) Appl. No.: 15/260,546

(22) Filed: Sep. 9, 2016

Related U.S. Application Data
(60) Provisional application No. 62/219,182, filed on Sep. 16, 2015.

(51) Int. Cl. A61F 2/08; A61B 17/06; A61B 17/17

(52) U.S. Cl. CPC A61F 2/0811; A61B 17/1714; A61B 17/06166; A61F 2002/0882; A61B 2017/0464

(57) ABSTRACT

Examples of the invention relate to transosseous suture constructs and methods for forming surgical attachments to a bone.

### FIELD OF THE INVENTION

Examples of the invention relate to transosseous suture constructs and methods for forming surgical attachments to a bone.

### BACKGROUND

A variety of surgical procedures require the attachment of something relative to a surgical site. For example, in surgery relating to the skeletal system, it is often advantageous to attach soft tissue, suture, implants, and/or other items in or adjacent to a bone. For example, soft tissues such as ligaments, tendons, fascia, other capsular material, and/or muscle may be attached to a bone. Such soft tissues may be adjacent bones at skeletal joints including but not limited to the joints of the hands and feet, ankle, wrist, knee, elbow, hip, shoulder, and spine. For example, it is often advantageous to pass a suture through a portion of a bone to form a transosseous attachment to the bone.

### SUMMARY

Examples of the invention relate to transosseous suture constructs and methods for forming surgical attachments to a bone.

In one example of the invention, a method of transosseous fixation of soft tissue to bone includes forming a first tunnel through the bone, the first tunnel having first and second spaced apart openings at an outer surface of the bone; forming a second tunnel through the bone, the second tunnel having third and fourth spaced apart openings at an outer surface of the bone; extending first and second suture limbs through the first tunnel, each of the first and second suture limbs having a first portion extending from the first opening and a second portion extending from the second opening; extending third and fourth suture limbs through the second tunnel, each of the third and fourth suture limbs having a first portion extending from the third opening and a second portion extending from the fourth opening; passing the second portion of each of the first, second, third, and fourth suture limbs through the soft tissue to form a simple stitch; securing the first portion of each of the first and second suture limbs and the second portion of each of the first and third suture limbs together; and securing the first portion of each of the third and fourth suture limbs and the second portion of each of the second and fourth suture limbs together.

In another example of the invention, a method of transosseous fixation of soft tissue to bone includes forming a first tunnel through the bone, the first tunnel having first and second spaced apart openings at an outer surface of the bone; forming a second tunnel intersecting the first tunnel, the second tunnel having a third opening at an outer surface of the bone; extending a first suture between the first and second openings, the first suture having a first portion extending from the first opening and a second portion extending from the second opening; extending a second suture between the first and third openings, the second suture having a first portion extending from the first opening and a second portion extending from the third opening; passing the second portion of each of the first and second sutures through the soft tissue to form a simple stitch; and securing the first portion of each of the first and second sutures and the second portion of each of the first and second sutures.

In another example of the invention, a method of transosseous fixation of soft tissue to bone includes forming a first tunnel through the bone, the first tunnel having first and second spaced apart openings at an outer surface of the bone; forming a second tunnel intersecting the first tunnel, the second tunnel having a third opening at an outer surface of the bone; forming a third tunnel intersecting the first tunnel, the third tunnel having a fourth opening at an outer surface of the bone; forming a fourth tunnel intersecting the first tunnel, the fourth tunnel having a fifth opening at an outer surface of the bone; extending a first suture between the first and second openings, the first suture having a first portion extending from the first opening and a second portion extending from the second opening; extending a second suture between the first and third openings, the second suture having a first portion extending from the first opening and a second portion extending from the third opening; extending a third suture between the first and fourth openings, the third suture having a first portion extending from the first opening and a second portion extending from the fourth opening; extending a fourth suture between the first and fifth openings, the fourth suture having a first portion extending from the first opening and a second portion extending from the fifth opening; passing the second portion of each of the first, second, third and fourth sutures through the soft tissue to form a simple stitch; and securing the first and second portions of each of the sutures.

In another example of the invention, a method of transosseous fixation of soft tissue to bone includes forming a first tunnel through the bone, the first tunnel having first and second spaced apart openings at an outer surface of the bone; forming a second tunnel intersecting the first tunnel, the second tunnel having a third opening at an outer surface of the bone; forming a third tunnel through the bone, the third tunnel having fourth and fifth spaced apart openings at an outer surface of the bone; forming a fourth tunnel intersecting the third tunnel, the fourth tunnel having a sixth opening at an outer surface of the bone; extending a first suture between the first and second openings, the first suture having a first portion extending from the first opening and a second portion extending from the second opening; extending a second suture between the first and third openings, the second suture having a first portion extending from the first opening and a second portion extending from the third opening; extending a third suture between the fourth and fifth openings, the third suture having a first portion extending from the first opening and a second portion extending from the third opening; extending a fourth suture between the fourth and sixth openings, the fourth suture having a first portion extending from the first opening and a second portion extending from the third opening; passing the second portion of each of the first, second, third and fourth sutures through the soft tissue to form a simple stitch; and securing the first and second portions of each of the sutures.

In another example of the invention, a method of transosseous fixation of soft tissue to bone includes forming a first tunnel through the bone, the first tunnel having first and second spaced apart openings at an outer surface of the bone; forming a second tunnel intersecting the first tunnel, the second tunnel having a third opening at an outer surface of the bone; extending first and second sutures between the first and second openings, the first and second sutures each having a first portion extending from the first opening and a second portion extending from the second opening; extending third and fourth sutures between the first and third openings, the third and fourth sutures each having a first portion extending from the first opening and a second portion extending from the third opening; passing the second portion of each of the first, second, third and fourth sutures through the soft tissue to form a simple stitch; and securing the first and second portions of each of the sutures.

In another example of the invention, a method of transosseous fixation of soft tissue to bone includes forming a portal extending from a location outside of a patient's body to a surgical site adjacent the bone and soft tissue, tying suture portions together to form a knot outside of a patient's body; and pulling the knot through the portal to the surgical site to form a mattress stitch.

### BRIEF DESCRIPTION OF THE DRAWINGS

Various examples of the present invention will be discussed with reference to the appended drawings. These drawings depict only illustrative examples of the invention and are not to be considered limiting of its scope.

FIGS. 1-25 are perspective views of a human shoulder illustrating transosseous suture constructs and methods according to examples of the present invention.

### DESCRIPTION OF THE ILLUSTRATIVE EXAMPLES

The following illustrative examples depict transosseous suture constructs and methods to anchor a member to a bone. The illustrative examples depict positioning a round suture in a bone tunnel to attach soft tissue to the bone. However, the transosseous suture constructs and methods of the present invention may be used to anchor other elements to a bone and the constructs may include round sutures, suture tapes, cables, soft tissues, grafts, and other members. The terms “suture” and “suture strand” are used herein to mean any flexible strand or other flexible member, natural or synthetic, able to be routed through a bone tunnel and through soft tissue and useful to secure the soft tissue to the bone. While the illustrative examples depict the attachment of the soft tissue of the rotator cuff to a humeral bone, it will be understood that instruments and methods according to the present invention may be used to anchor any member in any bone, at surgical sites anywhere in a patient's body, and for any purpose. The term “transverse” is used herein to mean to cross at an angle; i.e. not parallel. The term includes, but is not limited to, right angles.

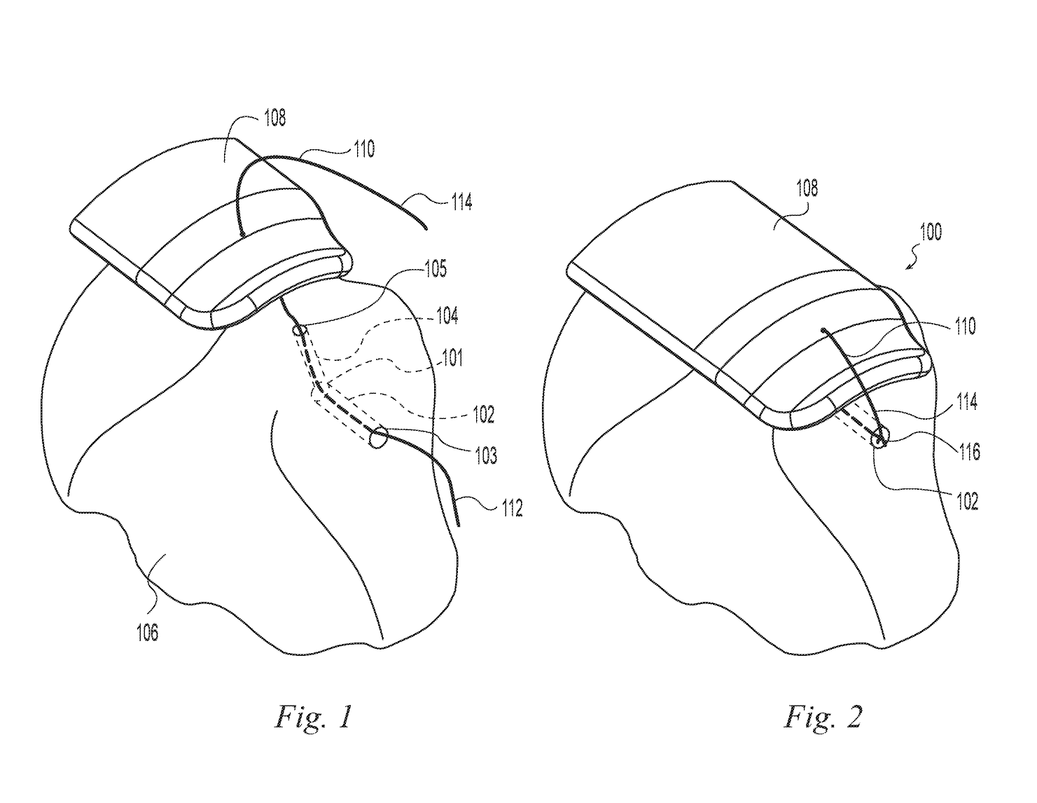

FIGS. 1-25 depict illustrative examples of transosseous suture constructs and methods. A simple construct 100 is depicted in FIGS. 1 and 2. Referring to FIG. 1, a transosseous tunnel 101 has been formed through a bone 106. The tunnel may be formed as a single straight or curved tunnel or as intersecting tunnel portions as is known in the art. In the illustrative example of FIGS. 1 and 2, the tunnel includes first and second intersecting tunnel portions 102, 104 formed in the bone 106, such as for example a humeral bone, adjacent a torn or detached soft tissue 108, such as for example a portion of a rotator cuff. In the illustrative example of FIGS. 1 and 2, the first tunnel portion 102 is formed from a lateral aspect of the bone medially into the bone and forms a first lateral opening 103 of the transosseous tunnel 101. The second tunnel portion 104 is formed from a more medial-superior aspect of the bone inferiorly to intersect the first tunnel 102 and forms a second medial-superior opening 105 of the transosseous tunnel 101. Formation of the bone tunnels may be performed freehand or guided. For example, a guide with intersecting guide axes may be used to guide one or more hole forming tools as is known in the art. Preferably, the medial superior opening 105 is positioned in the medial-lateral direction at a location that will be covered by the soft tissue 108 when the soft tissue 108 is pulled down, or reduced, to its desired attachment location. A suture 110, having first and second portions 112, 114, is passed through the transosseous tunnel 101 so that it passes through the bone, i.e. the suture 110 is transosseous. The second portion 114 of the suture, such as for example an end or a loop, is passed through the soft tissue inferiorly to superiorly to form a simple stitch through the soft tissue 108. Preferably, the suture is passed through the soft tissue at a position offset medially from the second tunnel 104 entrance so that when the suture is tensioned the soft tissue 108 is moved laterally to, or if the soft tissue is separately manually reduced it is held at, the desired attachment location.

Referring to FIG. 2, the soft tissue 108 has been reduced and the suture 110 secured. The suture may be secured in any suitable way. For example, the portion of the suture that was passed through the soft tissue 108 may be inserted through the entrance of the first bone tunnel 102 and the first and second portions may be secured by inserting an interference anchor into the first tunnel 102 as is known in the art. Alternatively, the first and second portions may be passed through an anchor with a suture locking mechanism and the anchor inserted into the bone tunnel 101. For example, various interference screws and knotless anchors are marketed by a large number of orthopedic equipment suppliers that may be used to secure the suture portions. Alternatively, the suture portions 112, 114 may be tensioned and tied in a knot 116 as depicted in the illustrative example of FIGS. 1 and 2. The suture ends extending from the knot 116 may be trimmed or, alternatively, passed through the soft tissue at an additional location and secured with another anchor or knot. The suture construct 100 holds the soft tissue 108 in position so that healing may occur. The portion 114 of suture routed over the top of the soft tissue 108 provides a downward compression force holding the soft tissue 108 against the bone. The remaining examples depict sutures that are knotted to secure them. However, it is to be understood that in any of the illustrative examples, knotless fixation may be substituted for the tying of knots.

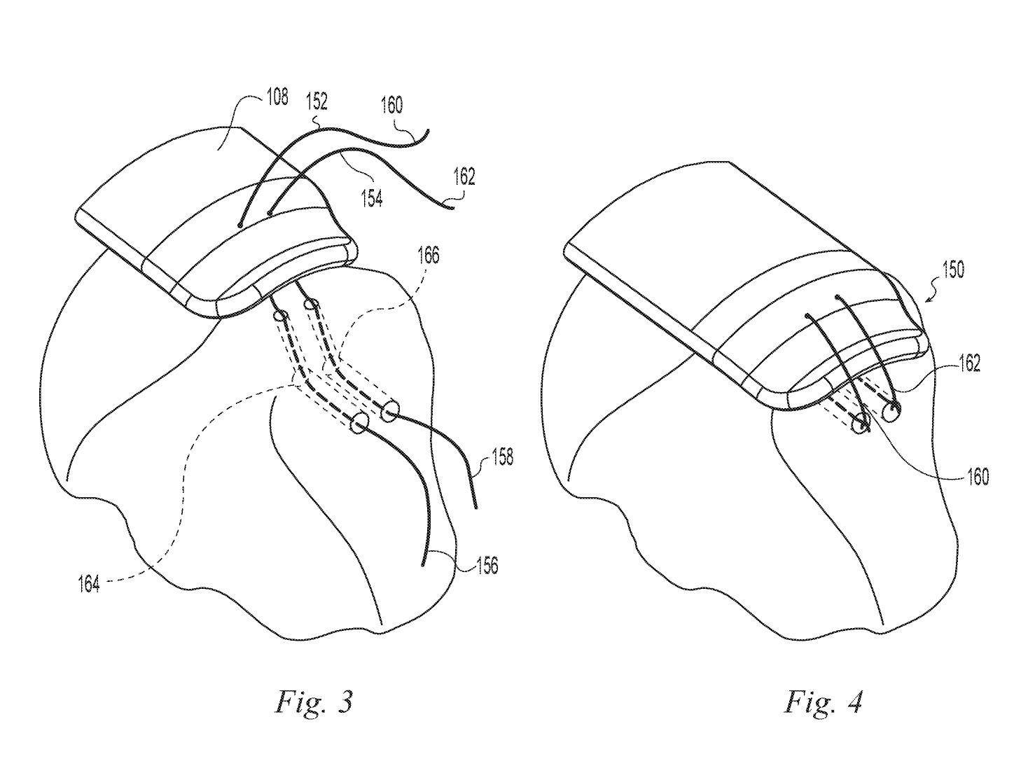

In FIGS. 3 and 4, two of the simple constructs 100 of FIGS. 1 and 2 have been combined to form a larger construct 150. In the illustrative example of FIGS. 3 and 4, two sutures 152, 154 having first portions 156, 158 and second portions 160, 162 are passed through two separate tunnels 164, 166 and through the soft tissue 108. In the illustrative example of FIGS. 3 and 4, the first and second portions of each suture are tied together to form simple stitches. However, other variations are possible. For example, the second portions 160, 162 may cross over one another and each may be secured with the first portion of the other suture. Similarly, the second portions 160, 162 may be tied together to form a mattress stitch before fastening the second portions 160, 162 to the bone.

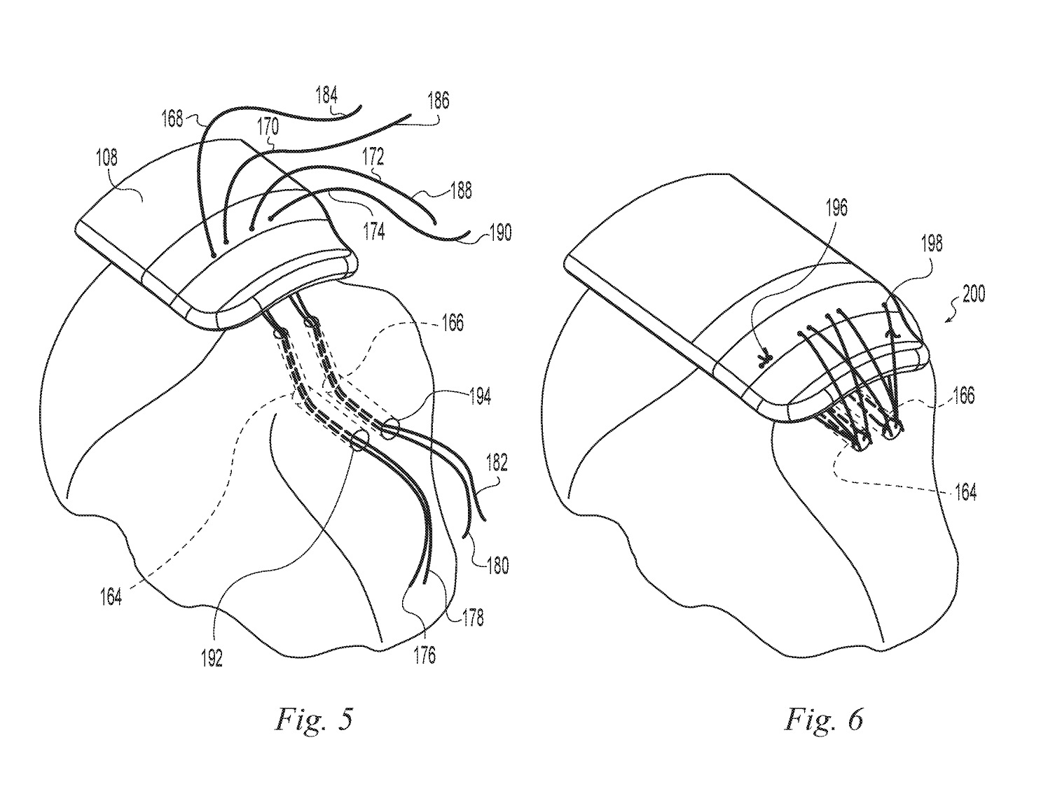

In FIGS. 5 and 6, the same tunnel configuration is used as in the construct 150 of FIGS. 3 and 4, however, two sutures 168, 170 are passed through the first tunnel 164 and two sutures 172, 174 are passed through the second tunnel 166 to form the illustrative construct 200. Each suture 168, 170, 172, 174 has respective first portions 176, 178, 180, 182 and second portions 184, 186, 188, 190. The second portions 184, 186, 188, 190 are passed through the soft tissue 108 at four spaced apart locations to spread the load on the tissue for better construct strength and to distribute the compression of the soft tissue against the bone. In the illustrative example of FIGS. 5 and 6, the second portion 184 of the first suture 168 is brought back to the lateral entrance 192 of the first tunnel 164. The second portion 186 of the second suture 170 is crossed over to the lateral entrance 194 of the second tunnel 166. The second portion 188 of the third suture 172 is crossed over to the lateral entrance 192 of the first tunnel 164. The second portion 190 of the fourth suture 174 is routed to the lateral entrance 194 of the second tunnel. The sutures are then secured as described previously. If the sutures are being knotted, they may be knotted at the tunnel entrances as shown or, for example, at any other location between the tunnel entrance and the location where the sutures pierce the soft tissue. Likewise, the sutures may be passed as simple stitches as shown in FIGS. 5 and 6 or one or more mattress stitches may be formed with combinations of the second suture portions. In the illustrative example of FIGS. 5 and 6, the free ends of the sutures are not trimmed after the initial suture fixation but are instead passed back through the soft tissue 108 to form first and second supplemental stitches 196, 198. The first supplemental stitch 196, for example, is formed by passing the free ends of the first and third sutures 168, 172 inferior-to-superior through the tissue and forming a mattress stitch. The second supplemental stitch 198, for example, is formed by passing one of the free ends of the second and fourth sutures 170, 174 inferior-to-superior through the tissue and tying the free ends of the second and fourth sutures 170, 174 to form a simple stitch. Since the first and second sutures 168, 170 pass through the same tunnel 164, there is a limit to the amount of space between the locations where they pass through the soft tissue beyond which the sutures may cause puckering of the soft tissue between them. The risk of tissue puckering is reduced by crossing the second and third sutures 170, 172 as shown in the illustrative example of FIGS. 5 and 6. For example, the sutures may be spaced inwardly beyond the edge of the bone tunnel from which they originate to increase their spacing from the first and fourth sutures 168, 174 respectively without the risk of puckering the tissue.

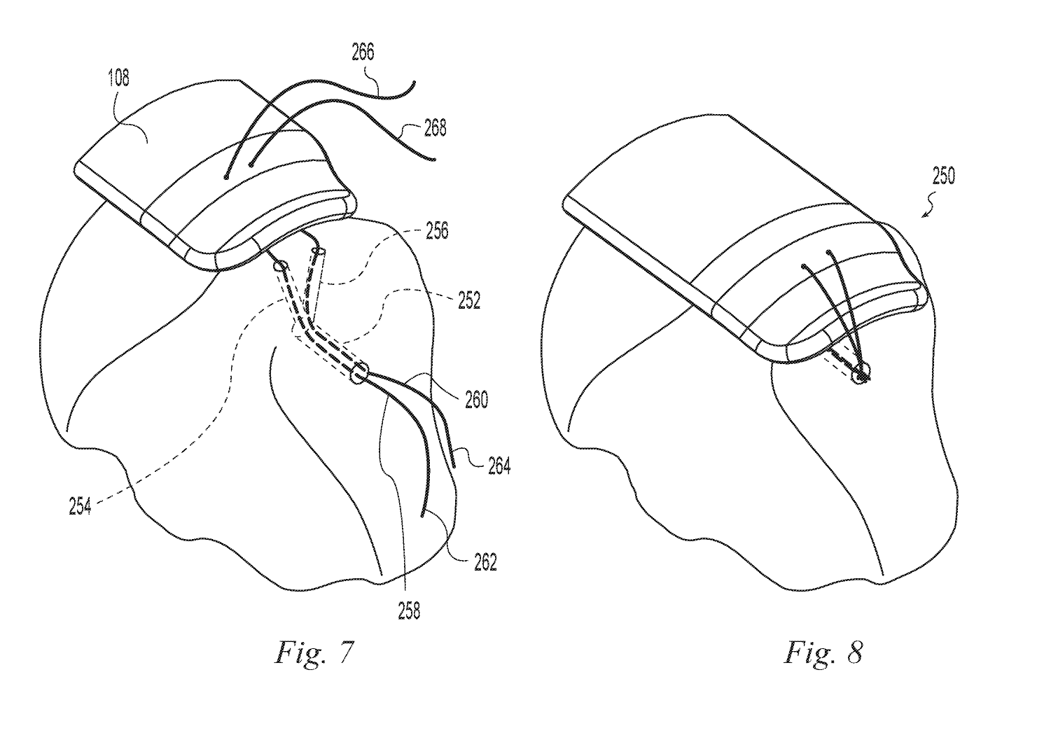

In FIGS. 7 and 8, first and second intersecting bone tunnels include a first tunnel portion 252 intersected by second and third tunnel portions 254, 256 such that the intersecting tunnel portions form a vee between them that diverges superiorly. In other words, a first transosseous tunnel is formed through the bone with a second tunnel intersecting it so that there are three openings at the surface of the bone. A first suture 258 is routed through the first and second tunnel portions 252, 254 and a second suture 260 is routed through the first and third tunnel portions 252, 256. The first and second sutures have respective first portions 262, 264 and respective second portions 266, 268. The second portions 266, 268 are passed through the soft tissue 108 inferiorly to superiorly at locations spaced apart approximately the same distance as the spacing between the openings to the second and third tunnel portions 254, 256. By varying the spacing between the second and third tunnel openings, constructs 250 with varying suture spacing may be accommodated without risk of puckering the soft tissue 108.

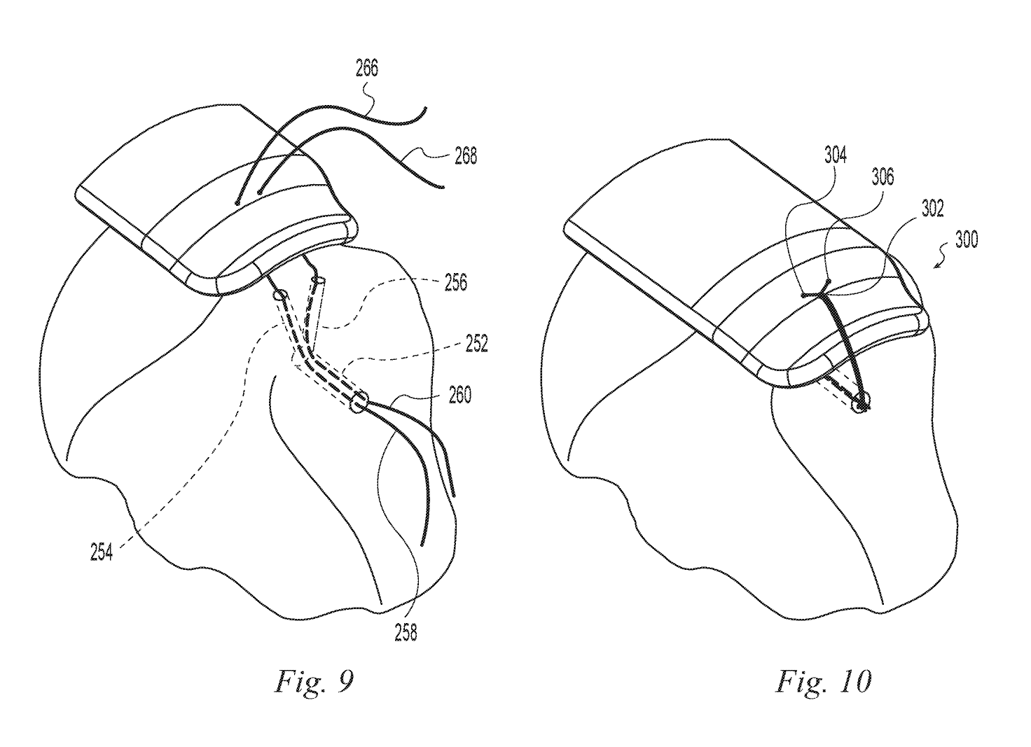

FIGS. 9 and 10 depict an illustrative example of a transosseous suture construct 300 similar to that of FIGS. 7 and 8 but with the second portions 266, 268 being tied together to form a mattress stitch before the sutures are secured to the bone. The mattress stitch may be formed by tying the knot 302 arthroscopically at the surgical site. However, due to the novel arrangement of the constructs of the present invention, the knot 302 may advantageously be tied outside the patient's body. For example, a portal may be formed that extends from a location outside of a patient's body to a surgical site adjacent the bone and soft tissue. The sutures may be tied together to form the knot 302 outside of the patient's body and the first portion of each of the first and second sutures may be pulled to move the knot through the portal to the surgical site. By selectively tensioning the sutures, the mattress stitch knot 302 may be selectively positioned horizontally anywhere between the locations 304, 306 where the sutures pierce the soft tissue and anywhere vertically between the opening to the first tunnel portion 252 and the piercing locations 304, 306.

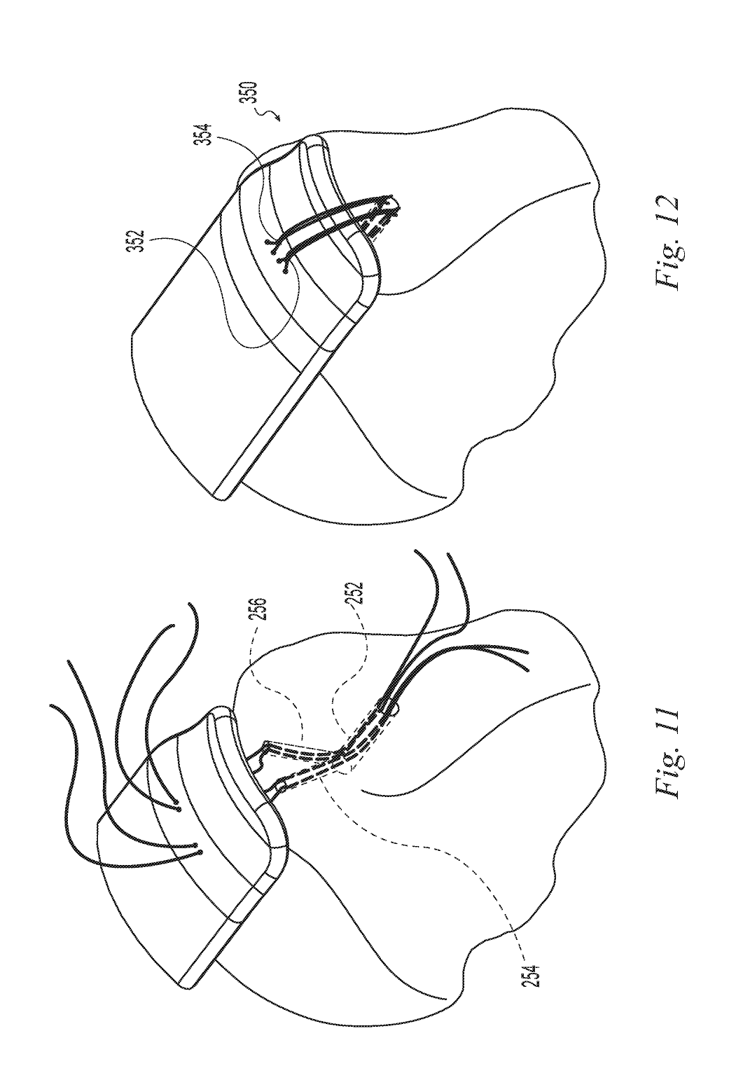

FIGS. 11 and 12 depict an illustrative example of a transosseous suture construct 350 similar to that of FIGS. 9 and 10 but with four sutures being routed through the first tunnel portion 252 and a first pair of the sutures passing through the second tunnel portion 254 and a second pair of the sutures passing through the third tunnel portion 256. The first pair of sutures is tied to form a first mattress stitch 352 and the second pair of sutures is tied to form a second mattress stitch 354.

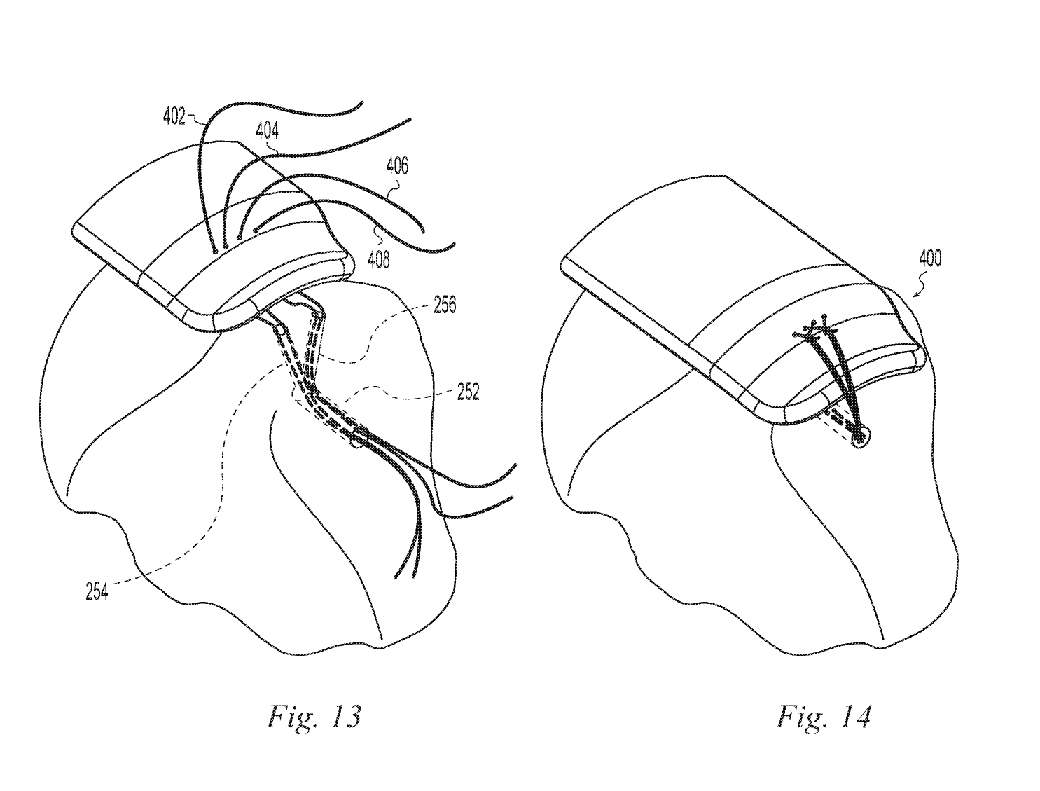

FIGS. 13 and 14 depict an illustrative example of a transosseous suture construct 400 similar to that of FIGS. 11 and 12 except that the mattress stitches are formed between first and third sutures 402, 406 and second and fourth sutures 404, 408 respectively such that the second and third sutures cross over to increase the compressive area of the construct and reduce the chance of puckering of the soft tissue.

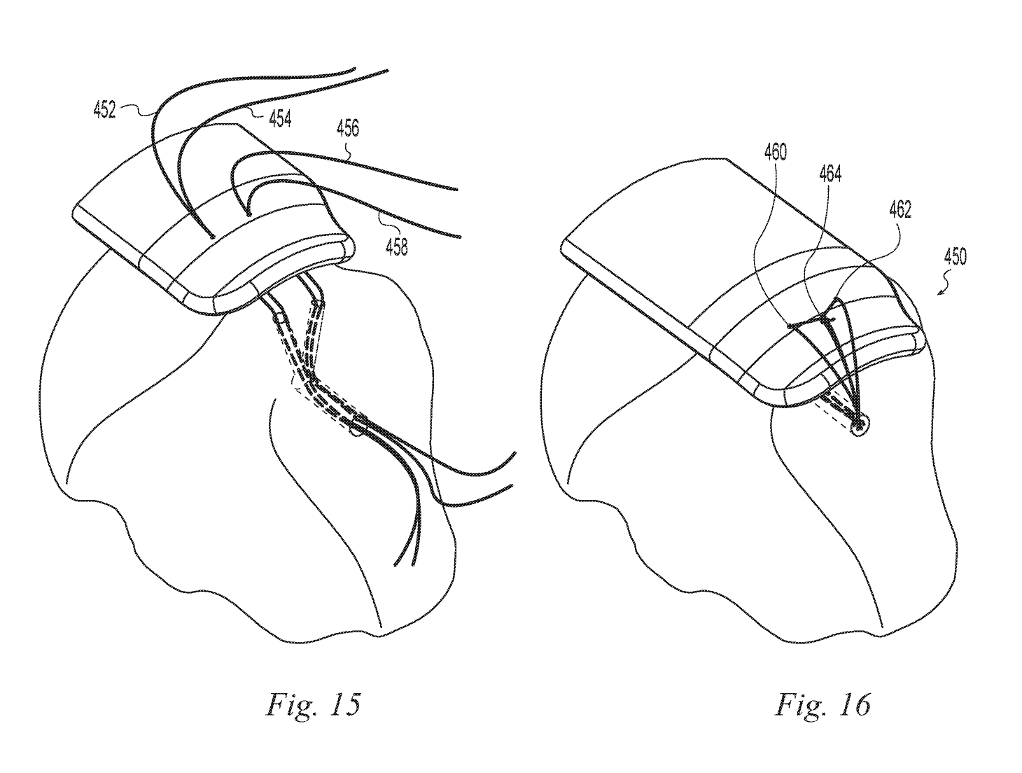

FIGS. 15 and 16 depict an illustrative transosseous suture construct 450 with the same tunnel configuration as FIGS. 7-14 and with four sutures routed in pairs through the second and third tunnel portions 254, 256 as in FIGS. 11-14. However, the first and second sutures 452, 454 are routed through the soft tissue together at a single location 460 rather than through separate locations. Likewise, the third and fourth sutures 456, 458 are routed through the soft tissue together at a single location 462 rather than through separate locations. A suture from the first pair of sutures is knotted with a suture from the second pair of sutures to form a mattress stitch 464. The knotted pair and the two remaining sutures, one from each of the original pairs, are then fixed to the bone.

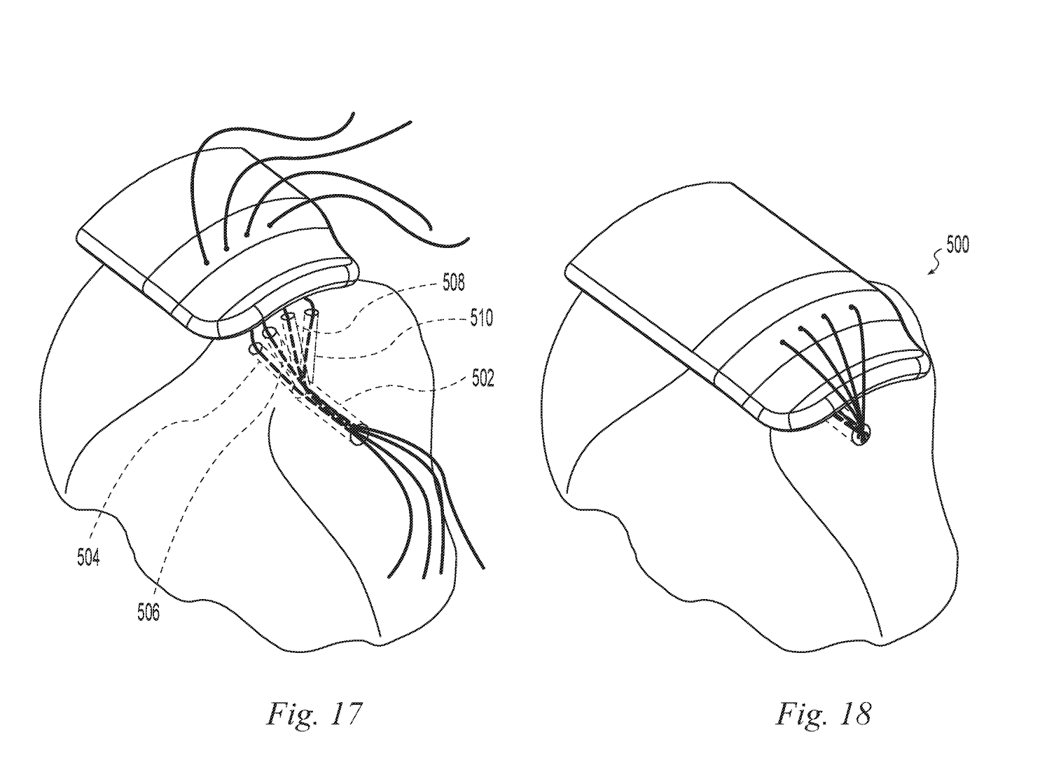

FIGS. 17 and 18 depict an illustrative transosseous suture construct 500 with more than two tunnel portions intersecting a first tunnel 502. In this example, second, third, fourth, and fifth tunnel portions 504, 506, 508, 510 intersect the first tunnel portion 502 and fan out over the superior surface of the bone. Four sutures are routed through the first tunnel portion 502 and one suture is routed through each of the intersecting tunnel portions. The sutures are passed through the soft tissue and secured to form simple stitches. Alternatively, any number of mattress stitches and/or suture crossings may be incorporated to enhance the strength and load distribution of the construct 500.

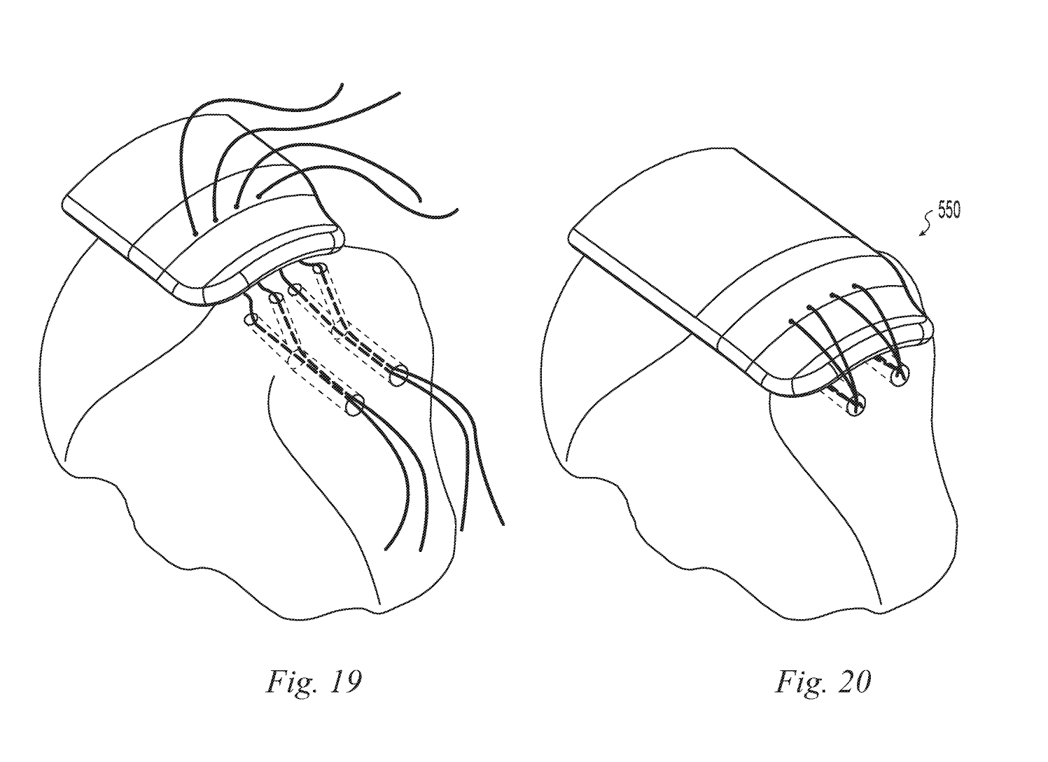

FIGS. 19 and 20 depict an illustrative transosseous suture construct 550 similar to that of FIGS. 7 and 8 but with two of the FIG. 8 constructs placed side-by-side.

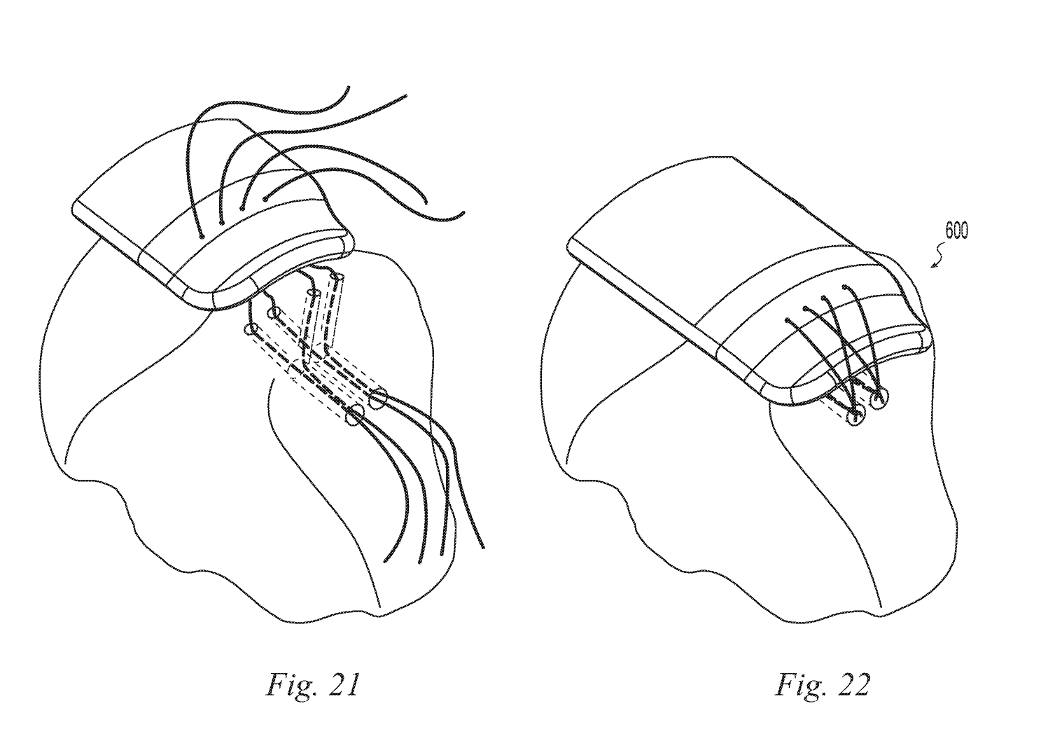

FIGS. 21 and 22 depict an illustrative transosseous suture construct 600 similar to that of FIGS. 19 and 20. However, in the construct 600 of FIGS. 21 and 22, the diverging intersecting tunnels of the second construct overlap the diverging tunnels of the first construct.

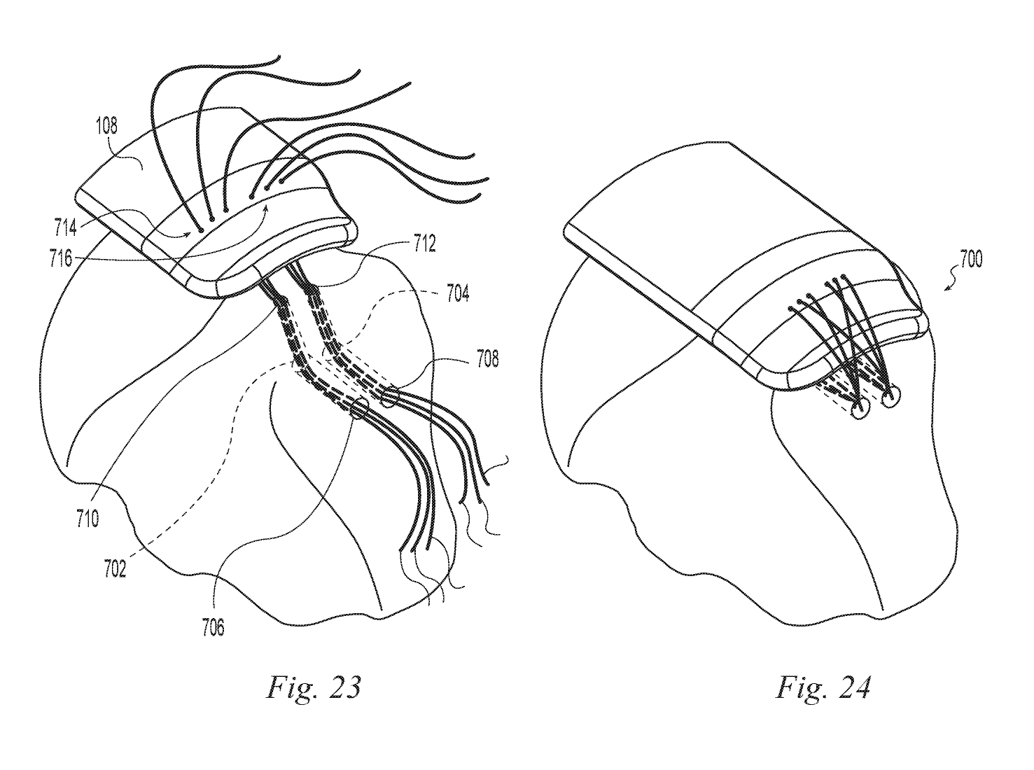

FIGS. 23 and 24 depict an illustrative transosseous suture construct 700 that uses the same tunnel configuration as in the example of FIGS. 3 and 4 and the example of FIGS. 5 and 6. In the example of FIGS. 23 and 24 three sutures are passed through each or two tunnels 702, 704. Each suture has a first portion exiting a lateral tunnel opening 706, 708 and a second portion exiting a medial tunnel opening 710, 712. The second portions are passed through the soft tissue in two groups of three 714, 716. Each group of three sutures is spaced apart in a row to spread the load on the tissue for better construct strength and to distribute the compression of the soft tissue against the bone. In the illustrative example of FIGS. 23 and 24, the first and third suture of each group is brought back to the lateral opening of the tunnel through which the suture was passed while the middle suture of each group of three is crossed over to the lateral opening of the other tunnel. The sutures are then secured as described previously. If the sutures are being knotted, they may be knotted at the tunnel entrances as shown or, for example, at any other location between the tunnel entrance and the location where the sutures pierce the soft tissue.

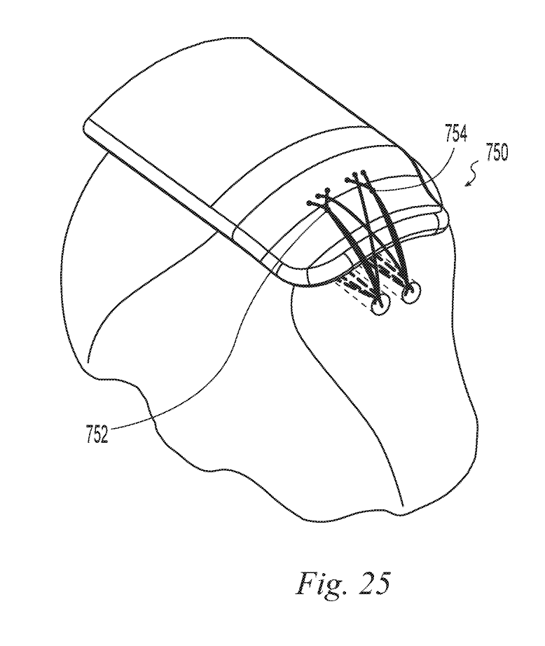

FIG. 25 depicts an illustrative transosseous suture construct 750 like that of FIGS. 23 and 24 except that the first and third suture in each group of three sutures 714, 716 are knotted together to form a mattress stitch 752, 754.

Various transosseous suture constructs have been presented to illustrate examples of the invention. The examples have included variations in the numbers of bone tunnels and tunnel intersections, the number of suture strands, the spacing of the sutures as they pierce the material being attached to the bone, the presence or absence of knots forming mattress stitches, and the presence or absence of suture crossings. It is to be understood that any number of tunnels, either straight or curved, whether intersecting or not may be advantageously used to form the novel transosseous suture constructs of the present invention. Likewise, any number of suture strands may be utilized and they may be combined in any configuration of number and placement within the various tunnels. Likewise, the piercing locations, various stitches, and suture crossings may be exchanged and combined among the depicted constructs to produce other permutations within the scope of the invention. Likewise, the sutures may be secured by knotting the sutures or by using knotless suture devices such as interference screws or suture anchors able to receive a portion of suture through the anchor and a member movable to lock the portion of suture relative to the anchor.

What is claimed is:

1. A method of transosseous fixation of soft tissue to bone comprising:
forming a first tunnel through the bone, the first tunnel having first and second spaced apart openings at an outer surface of the bone;
forming a second tunnel intersecting the first tunnel, the second tunnel having a third opening at an outer surface of the bone;
extending a first suture between the first and second openings, the first suture having a first portion extending from the first opening and a second portion extending from the second opening;
extending a second suture between the first and third openings, the second suture having a first portion extending from the first opening and a second portion extending from the third opening;
passing the second portion of each of the first and second sutures through soft tissue to form a simple stitch; and
securing the first portion of each of the first and second sutures and the second portion of each of the first and second sutures.

2. The method of claim 1 wherein securing the first portion of each of the first and second sutures and the second portion of each of the first and second sutures comprises securing the sutures together adjacent the first opening.

3. The method of claim 2 wherein securing the sutures comprises securing the sutures within the first tunnel.

4. The method of claim 1 wherein the first opening is on a lateral surface of a humerus and the second and third openings are on a more medial and superior surface of the humerus, further wherein passing the second portion of each of the first and second sutures through the soft tissue comprises passing the sutures inferior to superior through rotator cuff tissue, the method further comprising tensioning the suture limbs to move the rotator cuff tissue to a desired attachment location on the humerus.

5. The method of claim 1 further comprising tying the second portion of each of the first and second sutures together to form a mattress stitch prior to the securing step.

6. The method of claim 5 further comprising:
forming a portal extending from a location outside of a patient's body to a surgical site adjacent the bone and soft tissue;
tying the second portion of each of the first and second sutures together to form a knot outside of the patient's body; and
pulling the first portion of at least one of the first and second sutures to move the knot through the portal to the surgical site.

7. The method of claim 1 further comprising:
forming a third tunnel intersecting the first tunnel, the third tunnel having a fourth opening at an outer surface of the bone;
extending a third suture between the first and fourth openings, the third suture having a first portion extending from the first opening and a second portion extending from the fourth opening;
passing the second portion of the third suture through the soft tissue to form a simple stitch; and
securing the first and second portions of the third suture.

8. The method of claim 7 further comprising:
forming a fourth tunnel intersecting the first tunnel, the fourth tunnel having a fifth opening at an outer surface of the bone;
extending a fourth suture between the first and fifth openings, the fourth suture having a first portion extending from the first opening and a second portion extending from the fifth opening;
passing the second portion the fourth suture through the soft tissue to form a simple stitch; and
securing the first and second portions of the fourth suture.

9. A method of transosseous fixation of soft tissue to bone comprising:
forming a first tunnel through the bone, the first tunnel having first and second spaced apart openings at an outer surface of the bone;
forming a second tunnel intersecting the first tunnel, the second tunnel having a third opening at an outer surface of the bone;
forming a third tunnel through the bone, the third tunnel having fourth and fifth spaced apart openings at an outer surface of the bone;
forming a fourth tunnel intersecting the third tunnel, the fourth tunnel having a sixth opening at an outer surface of the bone;
extending a first suture between the first and second openings, the first suture having a first portion extending from the first opening and a second portion extending from the second opening;
extending a second suture between the first and third openings, the second suture having a first portion extending from the first opening and a second portion extending from the third opening;
extending a third suture between the fourth and fifth openings, the third suture having a first portion extending from the fourth opening and a second portion extending from the fifth opening;
extending a fourth suture between the fourth and sixth openings, the fourth suture having a first portion extending from the fourth opening and a second portion extending from the sixth opening;
passing the second portion of each of the first, second, third and fourth sutures through soft tissue to form a simple stitch; and
securing the first and second portions of each of the sutures.

10. The method of claim 9 wherein the second portions of the second and third sutures are crossed on top of the soft tissue.

11. The method of claim 9 wherein the second, third, fifth, and sixth openings form a row on the bone with the fifth opening between the second and third openings and the third opening between the fifth and sixth opening.

12. The method of claim 11 wherein the second portions of the second and third sutures are crossed on top of the soft tissue.

13. The method of claim 9 further comprising:
forming a portal extending from a location outside of a patient's body to a surgical site adjacent the bone and soft tissue;
tying at least two of the second portions together to form a knot outside of the patient's body; and
pulling at least one of the first portions to move the knot through the portal to the surgical site.

14. A method of transosseous fixation of soft tissue to bone comprising:
forming a first tunnel through the bone, the first tunnel having first and second spaced apart openings at an outer surface of the bone;
forming a second tunnel intersecting the first tunnel, the second tunnel having a third opening at an outer surface of the bone;
extending first and second sutures between the first and second openings, the first and second sutures each having a first portion extending from the first opening and a second portion extending from the second opening;
extending third and fourth sutures between the first and third openings, the third and fourth sutures each having a first portion extending from the first opening and a second portion extending from the third opening;
passing the second portion of each of the first, second, third and fourth sutures through soft tissue to form a simple stitch; and
securing the first and second portions of each of the sutures.

15. The method of claim 14 further comprising:
tying at least two of the second portions together to form a mattress stitch prior to the securing step.

16. The method of claim 14 wherein the second portions are passed through the soft tissue to form a row of four spaced apart piercing locations, the first piercing location in the row corresponding to the first suture, the next piercing location corresponding to the second suture, the next piercing location corresponding to the third suture, and the next piercing location corresponding to the fourth suture, the method further comprising:
tying the second portion of each of the first and second sutures together to form a mattress stitch prior to the securing step; and
tying the second portion of each of the third and fourth sutures together to form a mattress stitch prior to the securing step.

17. The method of claim 14 wherein the second portions are passed through the soft tissue to form a row of four spaced apart piercing locations, the first piercing location in the row corresponding to the first suture, the next piercing location corresponding to the second suture, the next piercing location corresponding to the third suture, and the next piercing location corresponding to the fourth suture, the method further comprising:
tying the second portion of each of the first and third sutures together to form a mattress stitch prior to the securing step; and
tying the second portion of each of the second and fourth sutures together to form a mattress stitch prior to the securing step.

18. The method of claim 14 wherein the second portions of the first and second sutures are passed through the soft tissue together at a first location and the second portions of the third and fourth sutures are passed through the soft tissue together at a second location spaced from the first location, the second portions of each of the second and third sutures being tied together to form a mattress stitch prior to the securing step.

19. The method of claim 18 further comprising forming a portal extending from a location outside of a patient's body to a surgical site adjacent the bone and soft tissue, wherein tying the second portion of each of the second and third sutures together to form a mattress stitch comprises tying the second portion of each of the second and third sutures together to form a knot outside of the patient's body, the method further comprising pulling the first portion of each of the second and third sutures to move the knot through the portal to the surgical site.

20. The method of claim 14 further comprising:
forming a portal extending from a location outside of a patient's body to a surgical site adjacent the bone and soft tissue;
tying at least two of the second portions together to form a knot outside of the patient's body; and
pulling at least one of the first portions to move the knot through the portal to the surgical site.
